(19) United States
(12) Patent Application Publication
(10) Pub. No.: US 2005/0130295 A1
(43) Pub. Date: Jun. 16, 2005

(54) Multifunctional self-diagnostic device for in-home health-checkup

(76) Inventor: Raymond Zu Qiang Li, Diamond Bar, CA (US)

Correspondence Address: LAW OFFICES OF J.F. LEE, 17800 CASTLETON STREET, SUITE 383, CITY OF INDUSTRY, CA, 91748, US

(21) Appl. No.: 10/904,818

(22) Filed: Nov. 30, 2004

(30) Foreign Application Priority Data
Jun. 3, 2002 (CN) 02122243.6

(51) Int. Cl. C12Q 1/68; C12M 1/34

(52) U.S. Cl. 435/287.2

(57) ABSTRACT

A new kind of of multifunctional self-diagnostic device for in-home health-checkup is disclosed. Normal ranges for various test results are stored in the devices″s memory and users can easily tell whether their bio-chemical test response(s) fall within the normal range, and thus providing benefits of early in-home detection subclinical status, inexpensive ways of health checkup, convinient monitoring of diseaase progression, guidance to the application of needed medical treatment, and thus reaching the goal of enhancing the health welfare of the general population.

the present invention claims the priority filing date under Patent Cooperation treaty (PCT) of prior application by the same inventor, having the last date of Dec. 3, 2004 to enter into any additional filing of Chapter II processing under PCT; to wit: Priority Date: Chinese filing date of Jun. 3, 2002.

PCT filing date of may 28, 2003 (filed in US), PCT number: PCT/US03/16611.

PCT Chapter II date: Dec. 24, 2003 (filing of Demand for Int″l Prelim Exam)

### BACKGROUND OF THE INVENTION

The present invention relates generally to a health-checkup device. More particularly, the present invention helps to provide an in-home, multifunctional self-diagnostic checkup device that measures various kinds bio-chemical test responses from sampled bodily fluids such as urine, blood, saliva, etc.

According to a person″s health status, general population can be divided into three groups. The first group is the healthy group of people who, in general, do not feel any simptoms of disease, whose physical examination by doctors would not show abnormality, whose lab report of various bio-chemical test responses from their bodily fluids would not show abnormality and whose medical procedures would not show abnormal either. This is the healthy population, constituting about 25% of the total population in civilized society.

The second group of people is the unhealthy population, aka patient population, who, in general, feel they have at least one or more symptoms of disease, and whose medical reports would show some sustaining or continuous abnormality as reported by physical examination conducted by medical doctors or physicians, or there is some sustaining/continuous abnormality on one or more of the bio-chemical responses from tests conducted on their bodily fluids, or the medical procedure performed would show some sustaining/continuous abnormality. This second group of people needs constant medical treatment, constituting about 25% of the total population.

The third group of people is the subclinical population, who, in general, do not feel any simptoms of disease, but whose lab report of various bio-chemical test responses from samples of their bodily fluids, or whose physical examinations by medical doctors, or whose medical procedures performed would show some sustaining/continuous abnormality. this group of people constitutes about 50% of the total population.

to prevent the subclinical population from gravitating towards, or becoming, patient population, who undoubtedly consume a lot of social resources for medical diagnosis and treatment, early detection of subclinical status and diagnosis of potential disease, therefore, will be crucial to the society as well as the individuals affected.

Although regular health checkup is encouraged, and people are aware of the importance of such checkup, it remains a fact that people miss the early timing for medical examination/report to detect subclinical status, as a result, miss the best opportunity to cure some diseases at early stages, with relatively lower overall costs.

As an alternative to regular doctor″s visits, which are in no way econamical or convinient, people may resort to self-diagnostic measures and devices. for purpose of early detecttion and self-checkup, there are off-the-shelf test kits or devices for specific bio-chemical response, such as test kits for thyroid functionality and HIV virus. These test kits usually are scaled-down version of those used in hospitals. After using these test kits, in most situations, users do not get the test results unless they send the tested sample to designated test lab for analysis and get the test readout back. oftentimes, the users get numerical readouts of their test results without knowing the meaning of the figures or whether the numbers are within normal range or not.

The high costs of using several test kits and the complexity of the operation from using the kit to obtaining a meaningful test readout poses a great obstacle to the goal of consumers having easy access to early detection of subclinical status or abnormality.

### SUMMARY OF THE INVENTION

The use of present invention will help overcome the aforementioned problems of (1) high costs and inconvinience associated with hospital checkups and doctor″s visits, (2) high costs and inconvinience of off-the-shelf single test kits/devices, and (3) lack of multifunctional checkup for various bio-chemical test responses.

Present invention of multifunctional self-diagnostic device for in-home health-checkup is embodied by a portable case with a lid connected to the case by mechanical means such as hinges. Several slots inside the lid are allocated for test agent strips of various kinds, with each agent strip geared toward one specific bio-chemical test response.

Inside the case, there can be found: a photocolorimetry slot, opti-electrical system that transformes optical signals to electronic signals, electronic analysis system that test readout figures against normal figures stored in the memory of said electronic analysis system, display system having colored lights located inside the case for easy recognition of test results, and a input/control device (keyboard) located inside the case. Wires or system-bus are built-in to connect the opti-electrical system, electronic analysis system, display sistem and the input/control device.

The opti-electrical system contains a light source and a spherical integrator with filter lens. The spherical integrator has a photo-electronic tube that can transform optical signals into electronic signals.

The electronic analysis system is based on a microcomputer with A/D converter, CPU and numeric comparator implemented by either software or hardware components. Normal ranges for various bio-chemical test responses are stored inside the memory of the elctronic analysis system. When a test result is obtained from the numeric comparator and falls in the normal range for that specific bio-chemical response, the electronic analysis system will send a signal to the display system and cause green light to be lit up. Vice versa, if a test result falls outside of the normal range for that bio-chemical response, red light on the display system will be lit up.

A keyboard is used for input or control when using the multifunctional device of present invention. Users can select wich bio-chemical test response is desired by pressing the identified keys on the keyboard to selected the stored normal ranges of specific bio-chemical test response.

Test samples for using the multifunctional device of present invention include urine, blood, stool (feces), saliva or exhaled air. Test samples are then fed to the corresponding agent strip for bio-chemical reaction; said agent strip is then fed to the photocolorimetry for determination of reflection light intensity. Calculation based on the reflection light intensity is then used to determine the numeric bio-chemical response for that specific test, and thereafter, a comparison to determine whether that specific test response falls within the normal range.

The agent strip used by present invention is made up by chemical agents sandwiched by plastic base and nylon membrane, cut into strips. The chemical agents embedded in each strip is designed and clearly marked for only one specific bio-chemical test response. The simplicity of learning to use and ease of operation is thus achieved.

Instead of the numerical display of the test result, which tends to confuse everyday users unless they possess certain level of medical knowledge, the choice of green/red indicator lights would simplify the architecture of the system and the corresponding use, and would help to ensure more households can have easy access to early detection of subclinical status and checkup.

The advantage of present invention includes, at least, the following points: 1. The in-home health checkup device of present invention combines various kinds of testing in one unit, lowering the costs of doing same multiple tests on different machines or kits. The test results are displayed by easy cognizeable lights, instead of numerical representation, so that it″s easier for users to understand the meaning of the result.

2. Each agent strip is designed to be used only once, and for testing only one specific bio-chemical response. This makes it easier for in-home users. The costs of manufacturing the device of present invention is significantly lower than the cost of those complex-type machines/devices that test multiple bio-chemical responses at the same time. The simplified multiple testing mechanism will be widely accepted by most families and thus promote the general health status by having people engage in early self-diagnostics and detection of subclinical status.

3. The health checkup device of present invention can be applied to detect many diseases and/or subclinical status, as well as bio-chemical responses, such as urine protein testing, urine glucose testing, uro-acid (pH) testing, urine accult blood testing, uring vitamin C testing, urine white blood cell and nitrite testing, urine density testing, urine pregnancy hormone testing, blood glucose testing, blood fat and cholesterol testing, blood C-reactive protein testing, blood thyroid testing, fecal occult blood testing, fecal bilirubin testing, fecal bilinogenemia testing, saliva chemistry testing, and extended air pH testing.

### BRIEF DESCRIPTION OF DRAWINGS

The accompanying drawings, which are incorporated in and constitute a part of this specification, illustrate the preferred embodiment of the invention and together with the description, serve to explain the principles of the invention.

A brief description of the drawings as follows:

FIG. 1 shows the typical components of the multifunctional self-diagnostic device for in-home health-checkup in present invention.

FIG. 2 shows the structural implementation of the opti-electrical system of the multifunctional self-diagnostic device in present invention.

FIG. 3 shows the structural implementation of the photo-electronic tube, electronic analysis system and display system of the multifunctional self-diagnostic device in present invention.

FIG. 4 shows the electrical circuitry diagram of the multifunctional self-diagnostic device in present invention.

FIG. 5 shows the cross-section of a test strip used in the multifunctional self-diagnostic device of present invention.

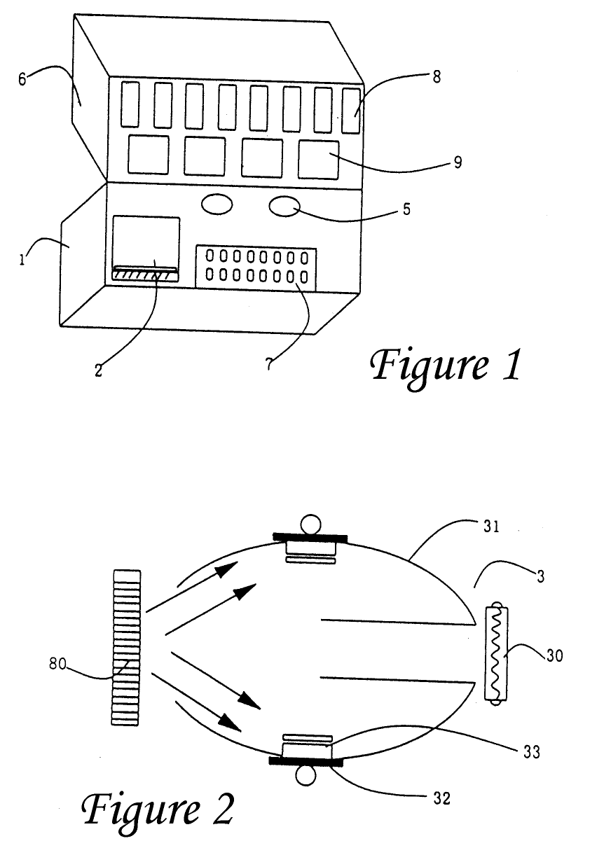

### DETAILED DESCRIPTION

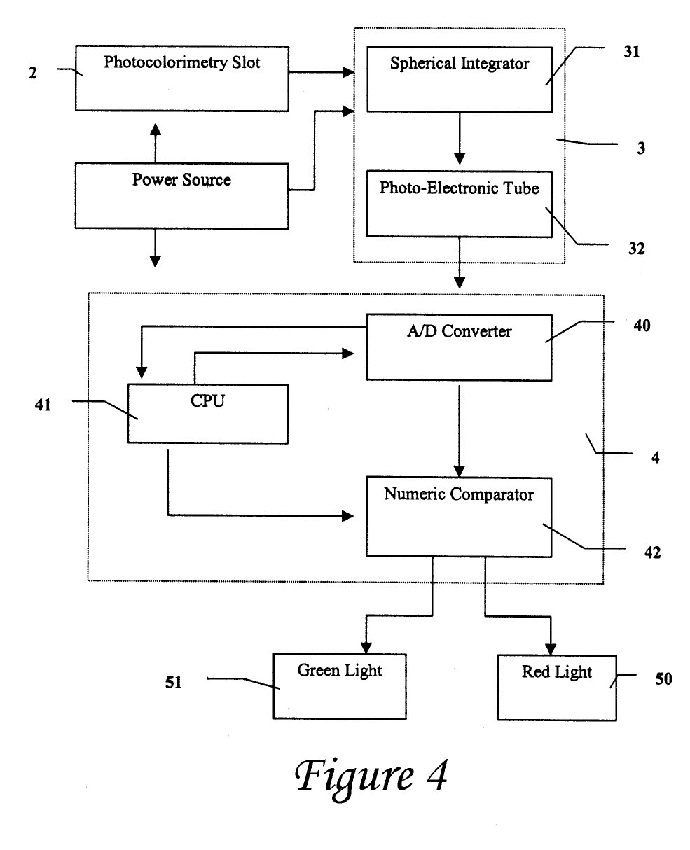

The multifunctional self-diagnostic device for in-home health-checkup of present invention, as shown in FOG. 1 through FIG. 4, includes a portable case 1, and lid 6 connected to said case 1 by hinges, a photocolorimetry slot 2, opti-electrical system 3, electronic analysis system 4, display system 5, keyboard 7, agent strip storage slots 8, and bio-chemical test agent storage slots 9. Agents strips and test agents for various bio-chemical tests are stored in slots 8 and 9.

Electrical and electronic wirings are provided to connect th opti-electrical system 3, the electronic analysis system 4 and the display system 5.

In FIG. 2, the implementation of opti-electrical system 3 is made up of a light source 30, a spherical integrator 31 with filter lens 33, and a photo-electronic tube 32 to transform light signal into electronic signal.

The electronic signal from the photo-electronic tube 32 is fed to the electronic analysis system 4, which then processed the result and then send corresponding signal to display system 5.

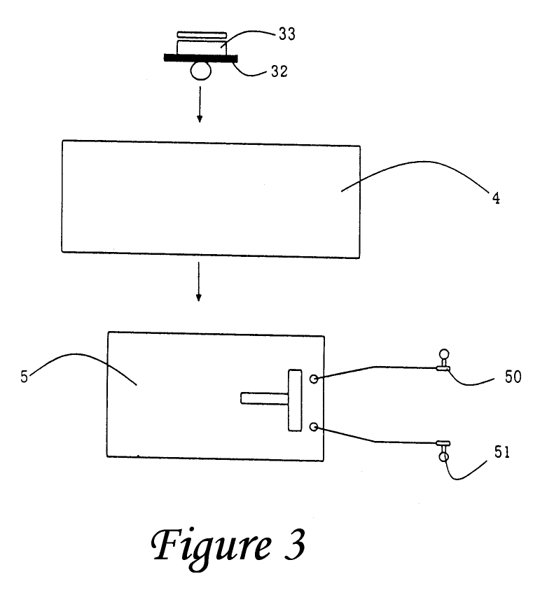

FIG. 3 and FIG. 4 show the construction of the electronic analysis system 4 by the use of a micro computer, including a A/D converter 40, CPU 41 and numerical comparator 42. Said A/D converter 40 uses the off-the-shelf product of AD574 produced by Analog Device, Inc. Similar converter chips also serve the same function. Said CPU 41 uses 8096 chipset by Intel, Inc. Said numeric comparator 42 uses Intel 767. Equivalent chips can be used. present invention will not elaborate the technical details of such off-the-shelf products.

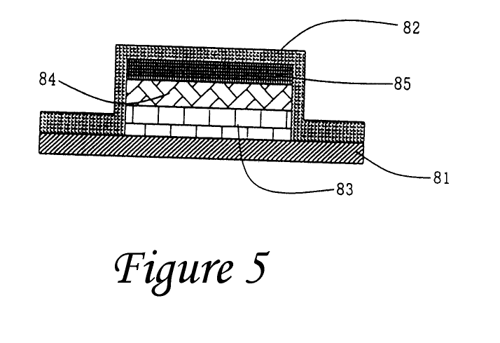

FIG. 5 shows the construction of agent strip used in the test device of present invention. Chemical agent for a single bio-chemical test response is embedded in one agent strip 80. Said agent strip 80 is made up of a plastic base 81 and a nylon membrane 82. In between the base 81 and the membrane 82, a water-absorbing layer 83, a test agent layer 84 and an iodate layer are embedded therein.

Display sistem 5 is attached to the case 1 and has color-coded lights for easy recognition by users. The color lights include red light 50 and green light 51. the two color lights are triggered by the results sent from the electronic analysis system 4 so that users can tell at a glance whether the result on a specific bio-chemical test response is normal. In addition to the use of lights, pre-recorded sounds or spoken words can be used to indicateed to users the result of a specific bio-chemical test response.

The implementation for the electrical and electronic connection among the aforesaid electronic analysis system 4, photo-electronic tube 32, light source 31, display system 5 is shown in FIG. 5, and can be done by people with average experience in industry. The test agent is the same as those commonly found in hospitals and doctors″ offices.

To use the self-diagnostic device in present invention, users can take a sample of, for example, his/her urine and dip the agent strip 80 (marked for testing specific bio-chemical response in urine) in the urine sample. the bio-chemistry ingredient to be tested (“target ingredient”) in the urine sample will have some kind of chemical/physical reaction with the test agent layer 84 inside the agent strip 80. Put the resulting agent strip in to photocolorimetry slot 2, and by the control of keyboard 7, light source 30 will shine a light on the agent strip, as shown in FIG. 2. Light reflected off agent strip 80 is received by spherical integrator 31.

The photo-electronic tube 32 on the spherical integrator 31 receives reflected light beams of two wave length: one beam of light whose wave length is made up of test light wavelength (the light from the dipped agent strip) and another beam of light whose wavelength is made up of reference light wavelength (the light selected by the keyboard for desired bio-chemical test response.) Photo-electronic tube 32 will convert the light signals received into electronic signals, and then send the elctronic signals to the electronic analysis system 4, for calculation of the rate of reflection. The terms and formula for calculation of rate of reflection is as follows: Tm: Reflection Light Intensity for Agent Strip to Test Wavelength Ts: Reflection Light Intensity for Agent Strip to Refernce Wavelength Cm: Reflection Light Intensity for Calibration Strip to Test Wavelength Cs: Reflection Light Intensity for Calibration Strip to Refernce Wavelength Formula of Rate: R=(Tm×Cs/Ts×Cm)×100% Lower reflection rate would be derived from the above formula when the target ingredient concentration is higher; and vice versa. The reflection rate is then compared to the normal range for testing on the target ingredient; the normal range for that specific bio-chemical test is stored in the memory of the electronic analysis system 4.

After the comparison, if the reflection rate for the target ingredient is within the normal range set for that specific bio-chemical test response, the green light 51 in the display 5 will go on. If, on the other hand, the reflection rate is outside of the normal range, then the red light 50 will light up.

The same operating procedure is applied to other bodily fluids in other bio-chemical test response.

1. A multifunctional self-diagnostic device for in-home health-checkup, comprising:
a. A case;
b. A lid detachable from said case with a plurality of slots for storing bio-chemical test agents and agent strips;
c. A photocolorimetry slot with automatic moving feeder inside said case;
d. An opti-electrical system that transforms optical signals to electronic signals;
e. An electronic analysis system that compares test readout figures against normal figures stored in the memory of said electronic analysis system;
f. A display system having green/red color lights for easy recognition of test result;
g. A keyboard device located inside said case for control and input;
h. A power-supply unit for home electricity voltage and means for connecting the electronic signal feeds among said systems and devise.

2. The self-diagnostic device of claim 1 wherein said lid is pivotably connected to said case.

3. The self-diagnostic device of claim 2 wherein said agent strip consists essentially of plastic base, nylon membrane, iodate layer, water-absorbing layer, and test agent layer for designated bio-chemical test response.

4. The self-diagnostic device of claim 3 wherein mormal ranges of designated bio-chemical test response are stored in said electronic analysis system built upon a microcomputer with A/D converter, CPU and numeric comparator implemented by either software or hardware components, so that in any designated bio-chemical testing, a first display signal for within-range test response will be generated, further causing the green light to be lit up, and a second display signal for outside-range response will be genrated, further causing the red light to be lit up.

5. The self-diagnostic device of claim 4 wherein said first or second display signal is derived from the comparator of said electronic analysis system having its numerical response fed from said opti-electronical system.

6. The self-diagnostic device of claim 5 wherein said firsst and second display signal will be used to trigger different lights and/or different sounds on said display sistem for easy recognition of test results.

7. The self-diagnostic device of claim 6 wherein said control and input device is a keyboard.
